(19) United States
(12) Patent Application Publication
(10) Pub. No.: US 2026/0283583 A1
(43) Pub. Date: Sep. 24, 2026

(54) System and Method for Compiling Standardized Radiological Equipment Testing Data

(71) Applicants: Ken Tirohn, Williamsville, NY (US); Ryan Erwin, WIlliamsville, NY (US)

(72) Inventors: Ken Tirohn, Williamsville, NY (US); Ryan Erwin, WIlliamsville, NY (US)

(21) Appl. No.: 19/169,311

(22) Filed: Apr. 3, 2025

Related U.S. Application Data
(60) Provisional application No. 63/678,578, filed on Aug. 2, 2024.

(51) Int. Cl. A61B 6/58; A61B 6/46

(52) U.S. Cl. CPC A61B 6/586; A61B 6/46; A61B 6/582

(57) ABSTRACT

A system and method for compiling standardized radiology equipment testing date, wherein the testing data is retrieved from diagnostic company databases. Radiologists and radiology centers are required to have their equipment tested and calibrated from time to time. Different diagnostic companies provide the test data in different formats. The system and method disclosed herein uses logic to transform the testing data into a standardized more usable format for radiologists and radiology centers.

### CROSS-REFERENCE TO RELATED APPLICATIONS

This application claims priority to U.S. Provisional Patent Application having Ser. No. 63/678,578, filed on Aug. 2, 2024, the entire disclosure of which is hereby incorporated herein by reference.

### BACKGROUND

Radiology diagnostic equipment requires regular testing and calibration to ensure accuracy and compliance with regulatory standards. In the context of testing and calibrating monitors used in radiological settings, monitor fidelity may drift, resulting in display inaccuracies. Consequently, radiology centers must implement quality assurance (QA) protocols to ensure all radiological diagnostic equipment and monitors are in compliance with all standards and to maintain certification. Conventionally, calibration must be conducted by a third-party, wherein the third-party is a diagnostic company, to ensure monitor accuracy. Each diagnostic company utilizes distinct proprietary data formats and methods for data collection, resulting in notable fragmentation in data handling. This fragmentation demands substantial manual effort from radiologists or support staff to compile and standardize information from multiple diagnostic companies into a consistent format suitable for regulatory reporting and compliance audits.

A system is required that facilitates secure connections with third-party management tools, employing encryption and authentication to ensure data privacy and integrity. Such a system should support multiple data formats and automatically detect and flag errors during data parsing and aggregation. It should also include a user interface allowing for manual correction of inconsistencies, generate audit logs for transaction traceability, and offer automated reporting capabilities for compliance and internal review. The system should be scalable and compatible with various proprietary systems to uniformly process and standardize data from different sources.

Typically, third-party diagnostic companies furnish diagnostic monitor data through websites or tools, enabling engineers to properly calibrate monitors. Each company provides unique tools that allow engineers to examine monitor-specific data and adjust monitors as necessary. Due to varied tools across third-party diagnostic companies, engineers must separately engage with each tool. Upon calibration, a compliance report is generated; however, report formatting varies between third-party diagnostic companies, complicating data management for radiologists or radiology centers with monitors from multiple vendors. A unified or standardized data format would streamline report integration into a single dashboard, allowing radiologists and radiology centers to access consolidated information through a single interface. It is noted that the third-party diagnostic company is a third-party conducting the testing, typically retaining diagnostic results and monitor data within their databases.

### FIELD

The present disclosure pertains to systems and methods for the standardized testing, calibration, and reporting of radiological diagnostic equipment, with an emphasis on secure data handling, error detection, standardization, and automated reporting.

### SUMMARY

In one aspect, the described technology relates to a system and method for compiling standardized radiological testing data. The system is designed to interact with various third-party management tools that store testing and calibration data related to radiology diagnostic equipment. It gathers, aggregates, and synthesizes data from multiple, non-standardized sources into a uniform format, thereby streamlining the periodic testing and calibration processes.

One objective of the technology is to facilitate secure connections with third-party management tools employing encryption and authentication methods to ensure data privacy and integrity. Another object is to standardize the aggregated data, making it recognizable and usable across the radiology industry for regulatory compliance and reporting purposes.

In an embodiment, the system employs a data scraping mechanism capable of logging into multiple third-party management tools hosting data regarding radiological equipment testing and calibration. It accesses these third-party management tools and extracts required testing data, regardless of the proprietary format or software used by the diagnostic companies.

In another embodiment, the system supports multiple data formats, including XML, CSV, and JSON. The extracted data is parsed and interpreted, then aggregated into a central repository. The system processes this aggregated data into a pre-defined standard format. Furthermore, an automatic error detection mechanism flags inconsistencies or missing data during parsing and aggregation stages.

In one aspect, the system includes a user interface that allows radiologists to manually correct flagged errors or inconsistencies. This interface ensures that any discrepancies in the data can be addressed before the data undergoes further processing or reporting.

Yet another object of the technology is to generate logs of all transactions for auditing purposes, ensuring transparency and traceability of the data. An automated reporting feature can be scheduled to run at predefined intervals, generating reports in the required format for compliance or internal review.

In another embodiment, the described system can be configured to operate on different types of databases. One embodiment integrates into an internally managed database owned and operated by the radiologist or radiology facility. Another embodiment operates on an external third-party management tool, managed by a diagnostic company, thereby allowing radiologists to access standardized data without extensive internal IT infrastructure.

The system is designed to be versatile and compatible with various proprietary systems used by different diagnostic companies. It ensures that data can be uniformly processed and standardized regardless of the testing company, supporting different implementation technologies to pull data from web-based platforms or direct database connections.

In one aspect, the detailed workflow includes securely logging into each third-party management tools, extracting pertinent testing and calibration data, parsing and interpreting the raw data, and aggregating the data into a central repository. The system then standardizes the aggregated data into a pre-defined standard format, flags any errors, and provides an interface for correction. Finally, the system exports the standardized data for reporting and regulatory compliance, and generates logs of all transactions for auditing purposes. The automated reporting feature can be scheduled to run at intervals to generate the necessary reports.

In another embodiment, the system is scalable to handle various data volumes and complexities based on the number of equipment units and frequency of testing and calibration. It offers multiple implementation options, whether as a local solution or a cloud-based service, ensuring flexibility for different sizes and scopes of radiology practices.

### BRIEF DESCRIPTION OF THE DRAWINGS

FIG. 1 is a flowchart illustrating the process of compiling radiological testing data through a sequence of steps involving user authentication, data retrieval, transformation, and display.

FIG. 2 is a block diagram illustrating a system architecture for compiling radiological testing data, comprising components such as API Gateway, AWS Fargate, Queue Service, and various data sources.

### DETAILED DESCRIPTION

The term “radiologist” as used herein and in the appended claims includes any hospital, radiology facility, radiology clinic, radiology center, or any other such facility or support personnel that uses radiology testing equipment that needs to be tested and calibrated to ensure compliance with radiology standards.

The term “radiology testing equipment” as used herein and in the appended claims refers refers to a range of specialized instruments used in medical imaging to diagnose and treat various conditions. The term may include X-ray machines, CT scanners, MRI machines, Ultrasound machines, nuclear medicine equipment, interventional radiology equipment, and radiotherapy equipment. In particular, the term includes the monitors and displays of each of these machines.

The term “diagnostic company” as used herein and in the appended claims refers to a company that provides radiology diagnostic equipment testing services to a radiologist.

The term “third-party management tool” as used herein and in the appended claims refers to a database owned by a diagnostic company, wherein the database contains the data resulting from testing of the radiology diagnostic equipment.

As discussed above, all radiologists must have quality assurance to make sure that their radiology diagnostic equipment, in particular, their monitors, are calibrated correctly and are certified for compliance purposes. The radiologist may use any diagnostic company to calibrate the radiology diagnostic equipment. The diagnostic company will provide a report with the specific date on any given piece of radiology diagnostic equipment. Each diagnostic company uses their own report with their own data. For this reason, when a radiologist uses several different diagnostic companies to calibrate their radiology diagnostic equipment, they will have several different reports. The present disclosure aims to take all of the reports and compile the date into a uniform dashboard for easy reporting. The radiologist will be able to log into one at-a-glance dashboard instead of several dashboards.

The system of the present disclosure includes the following components: front end; third-party management tools; Fetcher Server; Fetch API, Gateway API; Dynamo Database; Database Interface; and, Application Server.

### Front End

On the front end, a radiologist will sign up for a portal of the present disclosure and will select the manufacturer of the radiology diagnostic equipment that they have. The radiology diagnostic equipment will have been serviced by one or more diagnostic companies. The radiologist will specify which radiology diagnostic equipment data it would like and will enter the login credentials to that diagnostic company's management tool. The login credentials will then be stored securely. The system's dashboard will start out blank, but as the login credentials are provided and the system has access to the data from the respective databases, the dashboard will be populated.

### Third-Party Management Tools

The third-party management tools store all of the data from the testing and calibrating of the radiology diagnostic equipment. The database has login credentials specific to each radiologist. In most cases, radiologists will have radiology testing equipment from more than one diagnostic company, thereby requiring different diagnostic companies to test and calibrate the radiology testing equipment. Thus, the radiologist will likely have access to more than one third-party management tool.

### Fetcher Server

On the backend, a Fetcher Server containing fetch logic, will initiate the fetch logic to request, by a Fetch API, the inputted login credentials from the radiologist. The login credentials will be used to login into the selected third-party dmanagement tool. Once access is granted to the selected third-party management tool, an automated agent will open a web browser and will open all of the data about all of the radiology diagnostic equipment. The Fetcher Server will see the data, (as displayed on the page or through the raw data returned as undecorated data), store the data, and then in a later step, the system will transform the data so that it is uniform across all display types. In a preferred embodiment, the Fetcher Server is an AWS® Fargate Server.

The system is able to see the data, store the data, and transform the data so that it is in a uniform or standardized format. This will allow the radiologist to see all of their data on one screen, rather than having to go back to different screens and different logins. In some cases, the data maybe visually displayed in tables or graphs. In one embodiments the data is encapsulated into JSON.

In further embodiments, the system may provide additive logic, wherein functionality is provided to the radiological center by providing it with an option to tag displays by hospital or radiological center location.

### API Gateway

The system of the present invention includes an API Gateway. The function of the API Gateway is to provide request management. It receives Fetch API calls from clients and routes them to the appropriate backend service. The API Gateway also aggregates responses from multiple services into a single consolidated response from the client.

The API Gateway of the present system also includes protocol and data transformation. It translates between different protocols (HTTP to gRPC) or other data formats such as JSON to XML. In this way, the system enables compatibility between clients and services.

### Dynamo Database

After data is fetched from the selected third-party management tool, it is temporarily stored in a Database Interface before being stored in a Dynamo Database. The Dynamo Database is a durable location that prevents loss of data in the event of a server going down. The Dynamo Database will store the data until such time as it is requested by the Database Interface. After receiving a request from the Application Server, the Database Interface will retrieve the data from the Dynamo Database and will temporarily store the data before transferring it to the Application Server.

### Database Interface

As discussed above, there is a Database Interface that interfaces with the two servers, the Fetcher Server and the Application Server. As mentioned above, the Fetcher server requests and receives the data from the selected third-party management tool and then transfers the data to the Dynamo Database. The Database Interface also relays the request from the Application Server for data from the Dynamo Database.

The Database Interface temporarily stores the data as it is retrieved by the respective server. In the case of the Fetcher Server, the data is temporarily stored in the Database Interface before it is stored in the Dynamo Database. In the case of the Application Server, the Database Interface receives the request from the Application Server and then retrieves the data from the Database Dynamo, temporarily storing it before transferring it to the Application Server for transformation into the standardized data format.

The Database Interface is used for temporary security purposes and allows for secure access to the Dynamo Database. It filters out requests and securely accesses the Dynamo database without the Fetcher Server or the Application Server directly communicating with the Dynamo Database. This is done to prevent the Application Server or an application user from directly accessing the Dynamo database data. In this way, the application server is not able to directly communicate with the Dynamo database. This allows for the prevention of an application user directly obtaining access to the Dynamo database, thereby providing an extra layer of security.

### Application Server

The Application server includes transformation logic. There is transformation logic for each of the different diagnostic companies. The transformation logic generates the final JSON data.

The data is transformed by way of an Application Server that includes transformation logic. The transformation logic will provide mapping of the data from each of the diagnostic companies using a shared tenancy model. The data will then be transformed data that is in a standardized format. The data will be compiled into a single view. Visually, this may be a graph or a table. The data is encapsulated into JSON. In a preferred embodiment, the Application Server is an AWS® Fargate Server.

By including two servers, the Fetcher Server and the Application Server, each data source is able to rely on its own server, thereby reducing the amount of time to fetch the data. If there are two servers, each server can run in parallel. The Fetcher server exists for purposes of obtaining the raw untransformed data, while the application server generates the transformed final JSON data.

From the Application Server, the transformed data will be displayed on a device for the radiologist or other user to be able to view.

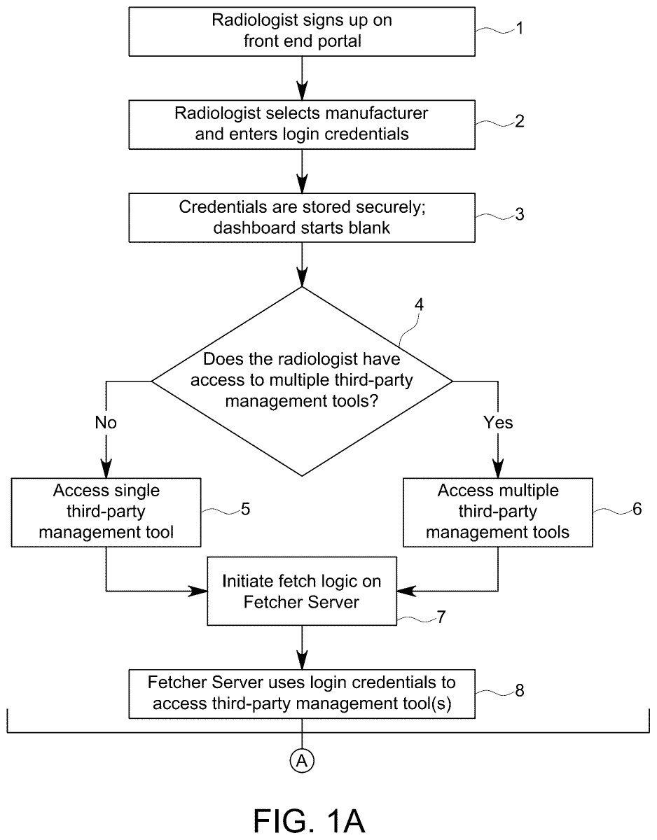

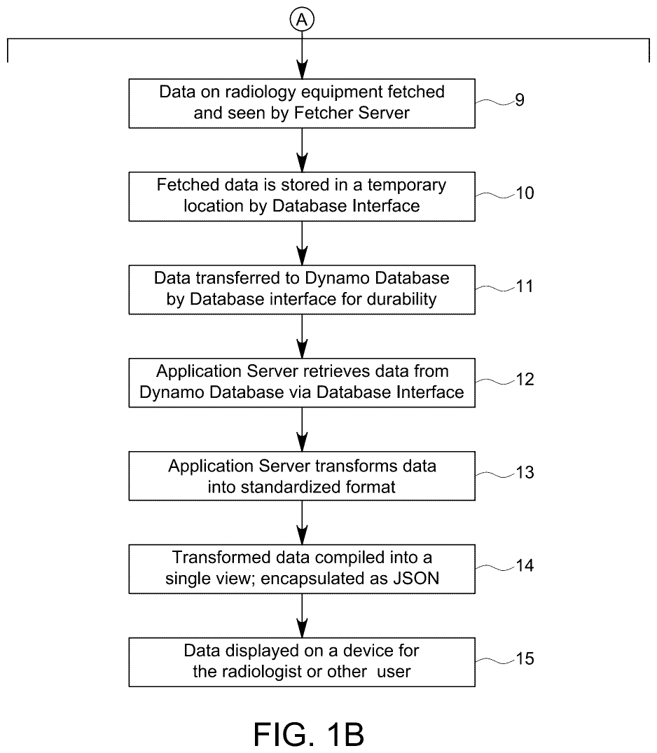

FIG. 1 shows a flowchart detailing a system for compiling radiological testing data. At step 1, a radiologist signs up on the front-end portal. Following this, in step 2, the radiologist selects a manufacturer and enters their login credentials. The credentials are stored securely, and a blank dashboard is presented, as depicted in step 3.

The process then evaluates whether the radiologist has access to multiple third-party management tools at step 4. If the radiologist has access to a single tool, step 5 entails accessing that one tool. Conversely, if multiple tools are accessible, step 6 describes accessing multiple third-party management tools.

Once access is determined, step 7 involves initiating fetch logic on the Fetcher Server. In step 8, the Fetcher Server utilizes the stored login credentials to access the third-party management tool(s). Subsequently, data on radiology equipment is fetched and seen by the Fetcher Server, as indicated in step 9.

In step 10, the fetched data is stored temporarily by the Database Interface. This data is then transferred to a Dynamo Database for durability, as shown in step 11. The Application Server retrieves the data from the Dynamo Database via the Database Interface in step 12.

The Application Server then transforms the data into a standardized format at step 13. In step 14, this transformed data is compiled into a single view and encapsulated as JSON. Finally, at step 15, the data is displayed on a device for the radiologist or another user.

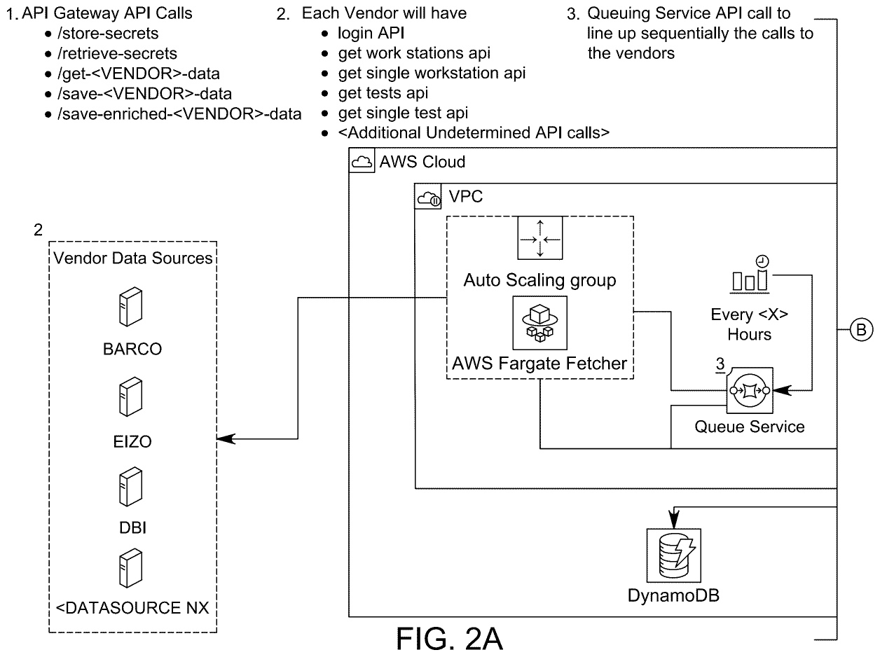

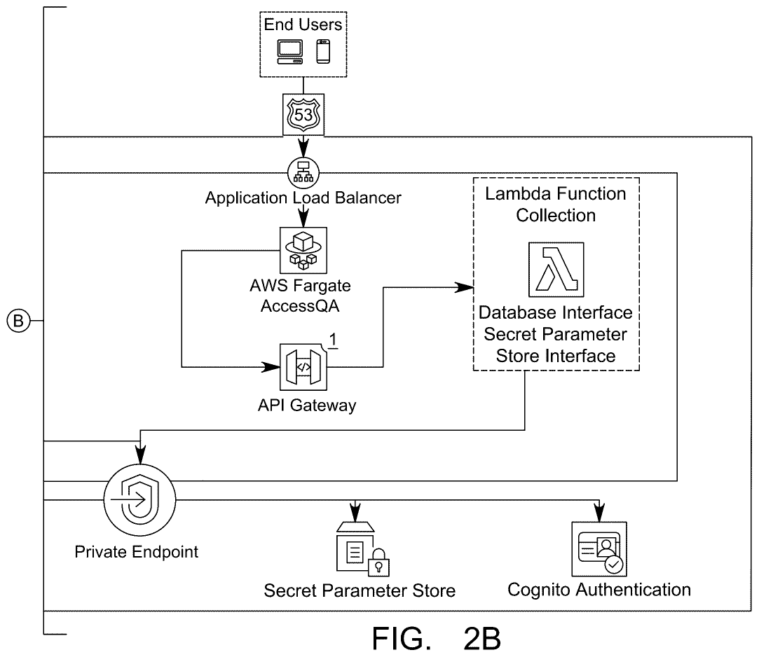

FIG. 2 shows a schematic overview of a data aggregation and standardization system for processing and managing radiology diagnostic equipment testing and calibration data. The system includes multiple components organized to interact seamlessly with third-party data sources, process the aggregated data, and present the standardized information to end users.

The system and method may be used in any setting where diagnostic equipment is tested and calibrated, including, but not limited to, radiology, cardiology, pathology, neurology, neurosurgery, ophthalmology, dentistry, surgery, or any other setting that uses diagnostic displays.

The system begins with the collection of data from various third-party data sources 2, represented as vendor data sources such as DBI, EIZO, and BARCO each housing specific diagnostic equipment data. These sources are integrated through a secure method using API gateway API calls 1, which handle different API endpoints for data retrieval, including GET/vendoR1-vendor3-data and similar calls. This gateway ensures secure and reliable extraction of data from the vendor systems.

The collected data is routed through a Virtual Private Cloud (VPC) to the AWS Services Group within the cloud infrastructure. Here, multiple components interact to process and secure the data. First, the data enters the API Gateway, which serves as an interface between the third-party sources and the internal processing system. The data then proceeds to AWS Restable Access-GW1, a RESTful service responsible for managing resource access and coordination.

The processed data is queued using a Queue service for sequential handling, maintaining data integrity and preventing bottlenecks. Event-driven processing is managed by Event 60 s timers to ensure timely data updates.

Security measures are enforced through Cognito Authentication to authenticate users or systems accessing the data. Sensitive information is securely stored in Secret Parameter Store, while the database variations, identified as Secret Parameter Store Variants, are logged to support storage and retrieval configurations.

For data analysis and aggregation purposes, the Lambda Function Collection component runs the necessary compute operations to standardize and aggregate the data into a consistent format. The results are stored in Application Level Selector, organizing data according to preset criteria.

End users access the standardized data through a graphical interface supported on various devices, depicted as computer, tablet, and mobile icons under the End users section. This interface allows radiologists and related personnel to view, manage, and extract the integrated data for reporting and compliance purposes.

Thus, the system integrates and processes complex datasets from multiple sources, incorporating robust security measures and user-friendly output methods, facilitating improved data governance in the context of radiology diagnostic equipment testing and calibration.

In one embodiment, a computer-generated method is disclosed comprising:
receiving from a parameter store database, by a login API, parameter data including login credentials;
receiving, by a fetch server, the parameter data;
responsive to receiving parameter data, the fetch server initiates fetch logic that includes the parameter data to request vendor data from a vendor database;
receiving from the fetch server, by a dynamo database, vendor data;
receiving, by an access API, vendor data from the dynamo database;
responsive to receiving vendor data from the access API, an access server initiates standardization logic to generate standardized data; and
presenting, by the access server, the generated standardized data for display on a device.

### EXAMPLES

### Example 1

In one illustrative example, a login API receives parameter data from a secret parameter store, wherein the data includes login credentials. In this example, the login credentials are owned by a radiologist or radiology center and include login credentials for third-party management tools. The third-party management tools include data from the results of testing radiology diagnostic equipment such as any diagnostic equipment and monitors. Each diagnostic company provides the test results data in a different format. Thus, this system and method transforms the testing data into a standardized form which is more useful for the radiologist or radiological center.

A fetch server will receive the parameter data from the login API and will initiate fetch logic that includes the parameter data to request vendor test results data from the third-party management tool. In a preferred embodiment, the fetch server is an AWS® Fargate system.

The vendor test results data is then received by a Dynamo DB. The vendor test results data is then received by an access API and responsive to receiving vendor test results data from the access API, an access server initiates standardization logic to generate standardized test results data. In one embodiment, the access server is an AWS® Fargate system.

The generated standardized test result data is presented by the access server for display on a device. The generated standardized test result data is available to an end user that is able to log into the system using data from a cognate authentication database by way of a database interface to login to an application load balancer to see the standardized test results date.

In one embodiment, the database interface is a secret store parameter store interface, which allows the API to communicate with the appropriate database in the database system. This system includes the dynamo database, the secret parameter store database, and the cognate authentication database.

It will be appreciated by those skilled in the art that while the system and method for compiling standardized radiological testing data has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

1. A system for compiling radiological testing data, the system comprising:
a. a front-end interface configured to receive login credentials from a user, the login credentials being associated with multiple third-party management tools each containing test data related to radiology diagnostic equipment;
b. a fetch server configured to utilize the received login credentials to access and retrieve test data from the third-party management tools;
c. an interface database configured to temporarily store the retrieved test data from the fetch server prior to processing;
d. an application server comprising transformation logic for converting the retrieved test data into a standardized data format;
e. a display device configured to present the standardized data to the user through the front-end interface.

2. The system of claim 1, wherein the transformation logic is capable of recognizing and converting various data formats including XML, CSV, and JSON into the standardized data format.

3. The system of claim 1, further comprising an API gateway configured to manage and route requests between the fetch server, the interface database, and the application server.

4. The system of claim 1, wherein the retrieve operation by the fetch server includes the use of a web browser automation agent to access the third-party management tools.

5. The system of claim 1, further comprising an error detection mechanism configured to identify and flag data inconsistencies during the transformation process.

6. The system of claim 1, further comprising a user interface configured to allow manual correction of flagged errors within the standardized data.

7. The system of claim 1, wherein the final standardized data format is encapsulated in JSON and stored in a durable storage.

8. A method for compiling radiological testing data comprising:
a. receiving, by a front-end interface, login credentials from a user;
b. utilizing, by a fetch server, the login credentials to access and retrieve test data from a third-party management tool;
c. temporarily storing the retrieved test data in an interface database;
d. converting the retrieved test data into a standardized format using transformation logic in an application server; and,
e. presenting the standardized data on a display device through the front-end interface.

9. The method of claim 8, further comprising automatically scheduling periodic data retrieval and processing operations to update the standardized data at predefined intervals.

10. The method of claim 8, wherein the transformation logic is executed in parallel for different diagnostic company data sources to optimize processing time.

11. The method of claim 8, further comprising generating logs of all transactions and data transformations for auditing and compliance purposes.

12. A computer-generated method for compiling radiological testing data comprising the steps of:
a. initiating, by a fetch server, fetch logic to request login credentials and radiology diagnostic equipment raw data;
b. receiving, by a fetch API, a request for the login credentials;
c. responsive to receiving, by a third-party management tool, the login credentials and the request for radiology diagnostic equipment raw data, the third-party management tool transmits the radiology diagnostic equipment raw data;
d. receiving, by the fetch server, radiology diagnostic equipment raw data;
e. receiving, by a database interface, radiology diagnostic equipment raw data;
f. receiving, by a dynamo database, radiology diagnostic equipment raw data;
g. receiving, by the database interface, a request for the radiology diagnostic equipment raw data, from an application server;
h. responsive to receiving, by the dynamo database, the request for the radiology diagnostic equipment raw data, the database dynamo database transmits the radiology diagnostic equipment raw data to the database interface;
i. responsive to receiving, by the application server, the radiology diagnostic equipment raw data, the application server initiates transformation logic;
j. transforming, by the application server, the radiology diagnostic equipment raw data into standardized data;
k. generating, by the application server, a graphical display of the standardized data; and
l. responsive to receiving, by a screen or display, the graphical display of the standardized data is displayed.

13. The computer-generated method of claim 12 wherein step b further includes an API gateway to route the request to the third-party management tool.
